(19) United States
(12) Patent Application Publication
(10) Pub. No.: US 2021/0379318 A1
(43) Pub. Date: Dec. 9, 2021

(54) DEVICE AND METHOD FOR DEACTIVATING AIRBORNE PATHOGENS

(71) Applicant: Advanced Imaging Research, Inc., Cleveland, OH (US)

(72) Inventors: Ravi Srinivasan, Beachwood, OH (US); Swathi R Srinivasan, Beachwood, OH (US)

(73) Assignee: Advanced Imaging Research, Inc., Cleveland, OH (US)

(21) Appl. No.: 16/896,809

(22) Filed: Jun. 9, 2020

(51) Int. Cl. A61M 16/10; A61M 16/20; A61M 16/00; A61M 16/06

(52) U.S. Cl. CPC A61M 16/1075; A61M 16/208; A61M 16/0003; A61M 16/0605; A61M 16/1055; A61M 2205/3368; A61M 2202/206; A61M 2202/203; A61M 2205/8206; A61M 2205/3673; A61M 16/107

(57) ABSTRACT

A breathing apparatus includes a first air pathway for receiving ambient air and channeling the air through a portion of the breathing apparatus, a heating section operatively coupled to the first air pathway and configured to elevate a temperature of the ambient air in the first air pathway to a first prescribed temperature, and a cooling section operatively coupled to the first air pathway and configured to reduce the temperature of the ambient air heated by the heating section to a second prescribed temperature, the second prescribed temperature lower than the first prescribed temperature. A breathing circuit is coupled to the first air pathway and configured to provide the cooled air to a user.

### TECHNICAL FIELD

The present invention relates to deactivating airborne pathogens, and, more particularly, to a device that removes and/or deactivates airborne pathogens, such as viruses, bacteria, germs and the like, and a method for performing the same.

### BACKGROUND ART

Inhalation of microbial aerosol particles can cause various health effects, ranging from moderate respiratory impairments to death. Studies have shown that large-scale infectious disease outbreaks, such as the outbreaks of severe acute respiratory syndrome (SARS) in 2003, influenza virus (swine flu H1N1) in 2009 and middle-east respiratory syndrome (MERS) in 2012 were triggered by airborne transmission of the viral agents (e.g., swine flu, bat corona, pig influenced viruses, etc.). At present, the coronavirus disease 2019 (COVID-19) continues to affect millions worldwide. Staying home, social distancing, washing hands with soap and water and wearing N95 facemasks in public, although effective, do not completely prevent disease spread and or protect healthcare workers and first responders that are exposed to infected patients and areas infected with COVID-19.

Virus reduction or inactivation has been demonstrated with chemical agents, ultra-violet (UV) radiation, non-thermal plasma (NTP) and other means, all which give rise to additional ozone generation that can be harmful to humans. In addition, UV radiation can damage the skin and cause skin cancer. NTP, on the other hand, is extremely complex and not portable. Further, chemicals cannot be used in inhalation devices.

Experiments performed by the World Health Organization (WHO) scientists have proven ways to dramatically reduce the virus count with virus cultures operating at elevated temperatures (see the World Health Organization report on First data on stability and resistance of SARS coronavirus compiled by members of WHO laboratory network, 2003. World Health Organization, Geneva, Switzerland. http://www.who.int/csr/sars/survival_2003_05_04/en/index.html). The WHO experiments analyzed the influence of temperature on a virus from −80 to 56° C. This was followed by a peer reviewed paper by Dr. Lisa Casanova reporting virus survival on surfaces at different temperatures and humidity. A recent, non-peer review paper titled “Evaluation of heating and chemical protocols for inactivating SARS-CoV-2” posted by Dr. Remi N. Charrel, et al. online on Apr. 11, 2020, reported dramatic virus life reductions at 56, 60 and 92° C., respectively (see also Doremalen et al. published by the New England Journal of Medicine Apr. 16, 2020 on the ‘Aerosol and Surface Stability of SARS-CoV-2 as Compared with SARS-CoV-1’ viruses (https.//doi.org/10.1101/2020.03.09.20033217). While it is known that elevated temperatures can reduce the life of an infectious disease, elevated room temperatures are impractical and unlivable.

UV C radiation between 100-280 nm wavelength has been used to irradiate viruses and is efficient for disinfection of food, water and beverage, hospital gowns, personal items (cell phones, masks, etc.), biomedical equipment, hospital rooms, buses, trains and airplanes (see, e.g., U.S. Pat. Nos. 9,895,458, 9,700,647, 9,706,794, 10,251,498, 10,245,341, 10,245,340, 10,639,390, 10,583,212 and 10,583,213). UVC light, however, is harmful for the human skin and can cause cancers. UVC light also produces ozone that can be harmful if inhaled in large amounts.

Atmospheric pressure, NTP applications are also used to inactivate airborne bacteria and viruses, and are typically used in food preservation, wound healing, animal husbandry, soil treatment, etc. (see, e.g., US Patent Publication Nos. 2020/0016286A1 and 2020/0071199A1). NTP employs high dielectric barrier discharge mechanisms and high AC voltages (e.g., on the order of 4-10 kV) that is intended to neutralize or stabilize the unstable airborne pathogens as they pass through a discharge tube filled with high-permittivity dielectric materials. As can be appreciated, carrying high voltage equipment on one's body can be dangerous. NTP also generates ozone, which is harmful. Thus, miniaturizing this complex equipment for personal use is not recommended.

U.S. Pat. No. 10,335,618 to Zhou discloses a button-cell battery operated facemask with integrated UV C light emitting diodes (LED's) that irradiate and deactivate incoming air. In such device the LED's must be intense to deactivate the airborne pathogens. Further, the cascade of LEDs are always ON in the serpentine air pathways, and this generates small amounts of ozone very close to the nasal cavity. No effort is made to filter the ozone with activated charcoal filter or the like before the irradiated air is inhaled by the subject.

US Patent Publication No. 2009/0112299 A1 to Chapman discloses a facemask that includes a therapeutic, battery powered, flexible heater that provides relief from symptoms of the common cold by maintaining air temperature over the nasal cavity and surrounding sinuses at roughly 46° C. for 20 minutes. This type of a facemask cannot be worn for hours at a time, as it may be harmful to the user's face, skin, nose, nasal cavity, sinuses, throat and upper chest.

U.S. Pat. No. 4,793,343 to Cummins discloses a facemask that includes an integrated battery-powered heater. The facemask reduces breathing discomfort in persons having respiratory and heart ailments by controlling the incoming air temperature between 10-27° C. The facemask of Cummins, however, clearly is not a pathogen deactivation device.

U.S. Pat. No. 4,620,537 to Brown discloses a moisture and heat exchanging facemask that is powered by an external source. The facemask minimizes heat expulsion during exhalation, thereby optimizing the amount of power drawn by the unit to maintain heat to the facemask. This locally-heated facemask and heat exchanger, however, is also not a pathogen deactivation device.

U.S. Pat. No. 5,511,541 to Dearstine discloses a warm air mask. This mask, however, suffers from the same issues referenced above with respect to Chapman, Cummins and Brown.

In summary, UVC radiation, atmospheric pressure NTP or warmed facemasks are neither useful nor beneficial for deactivating airborne pathogens for personal use. A methodology to deactivate pathogens, including, for example, SARS, the cold, flu, H1N1, MERS and COVID-19 viruses and the like, is needed to improve safety of healthcare providers and first responders attending to patients infected with a pathogen, such as a respiratory virus.

### SUMMARY OF INVENTION

A device and method in accordance with the invention deactivate airborne pathogens, such as viruses, germs, bacteria, and the like. More particularly, ambient air is collected and subjected to a temperature sufficiently high to deactivate the airborne pathogens in the collected ambient air. For example, raising the air temperature to 120 degrees C. or higher has been found to deactivate airborne pathogens. Prior to providing such heated air to a user, the heated air is cooled back down to a temperature that is near ambient air temperature. The cooled air then is provided to a user via a facemask or the like.

In some embodiments, the ambient air is filtered before and/or after being heated. Such filtering may be performed using a medical-grade micro-particulate filter. A filtering surface of the filter may be have a hydrophobic coating and/or coated with cationic moieties. The hydrophobic coating helps trap any remaining airborne liquid in the air, while the cationic moieties help trap any anionic ribonucleiacid (RNA) fragments generated due to the deactivation process. In some embodiments, air pathways may be formed from metals, such as copper or aluminum, which provide shielding and can assist in cooling the heated air.

The device and method in accordance with the invention can be used as a personal breathing apparatus that can be worn by a user (e.g. in a backpack or side mount configuration). Additionally, the device and method in accordance with the invention can be configured to supply conditioned air to buildings (e.g., homes, commercial buildings, and the like) and to vehicles (e.g., automobiles, trucks, busses, trains, aircraft and the like).

According to one aspect of the invention, a breathing apparatus for deactivating airborne pathogens includes: a first air pathway for receiving ambient air and channeling the air through a portion of the breathing apparatus; a heating section operatively coupled to the first air pathway and configured to elevate a temperature of the ambient air in the first air pathway to a first prescribed temperature that deactivates airborne pathogens; a cooling section operatively coupled to the first air pathway and configured to reduce the temperature of the ambient air heated by the heating section to a second prescribed temperature, the second prescribed temperature lower than the first prescribed temperature; and a breathing circuit coupled to the first air pathway and configured to provide the cooled air to a user.

In one embodiment, the breathing circuit comprises a facemask portion configured to cover a portion of a user's face, the facemask portion including a second air pathway configured to receive the air from the first air pathway that is cooled by the cooling section and provide the cooled air to at least one breathing port arranged to overlie a user's facial cavity.

In one embodiment, the facemask portion further includes: an exhaust port configured to vent exhaled air to the ambient environment; and at least one valve coupled to the second air pathway, the at least one valve configured to inhibit mixing of air exhaled by the user with air in the second air pathway.

In one embodiment, the facemask portion comprises a coupler operative to selectively couple the second air pathway to the first air pathway.

In one embodiment, the breathing apparatus includes a filter element arranged in the first air pathway upstream from the heating section, the filter element configured to filter the ambient air provided to the heating section.

In one embodiment, the breathing apparatus includes a filter element arranged in the first air pathway downstream from the heating section and upstream from the breathing circuit, the filter element configured to filter the air heated by the heating section.

In one embodiment, the filter element comprises a coating including cationic moieties.

In one embodiment, the filter element comprises a hydrophobic coating.

In one embodiment, the filter element comprises a micro-particle filter element configured to block 0.05 micron airborne pathogens.

In one embodiment, the heating section includes a heating surface over which the ambient air flows, the heating section configured to maintain a temperature of the heating surface within the range of 200 degrees C. to 250 degrees C.

In one embodiment, the first prescribed temperature is at least 190 degrees C.

In one embodiment, the second prescribed temperature is within 3 degrees C. of a temperature of the ambient air.

In one embodiment, the breathing apparatus includes a power supply electrically coupled to at least one of the heating section and the cooling section, the power supply operative to provide electric power to the at least one of the heating section and the cooling section.

In one embodiment, the power supply comprises a battery electrically coupled to at least one of the heating section and the cooling section.

In one embodiment, at least one of the first air pathway or the second air pathway comprises a metal inner liner.

In one embodiment, the metal inner liner is formed from copper or aluminum.

In one embodiment, the heating section comprises a fan operative to create a positive pressure from an input side of the heating section to an output side of the breathing circuit, and a temperature sensor operative to measure a temperature of air output by the heating section.

In one embodiment, the breathing apparatus includes a controller operatively coupled to at least one of the heating section and the cooling section, the controller configured to regulate at least one of a temperature of air output by the heating section and a temperature of air output by the cooling section.

In one embodiment, the cooling section comprises a heat expeller.

In one embodiment, the cooling section comprises a thermoelectric cooling module.

In one embodiment, the heating section comprises at least one of a resistor, a cartridge heating element, or a positive temperature heating coefficient heating element.

In one embodiment, the facemask portion comprises a securement strap for securement of the facemask portion to the user's face.

According to another aspect of the invention, a method of conditioning air provided to a user includes: collecting air from the ambient environment; heating the collected ambient air to a first prescribed temperature, the first prescribed temperature sufficient to deactivate airborne pathogens; cooling the heated air to a second prescribed temperature, wherein the second prescribed temperature is lower than the first prescribed temperature; and providing the cooled air to the user.

In one embodiment, providing the cooled air to the user includes providing the cooled air to a facemask worn by the user.

In one embodiment, the method includes using a hydrophobic filter element to filter air provided to the user.

In one embodiment, the method includes using a filter element coated with cationic moieties to filter air provided to the user.

In one embodiment, the first prescribed temperature is at least 190 degrees C.

In one embodiment, the second prescribed temperature is within 3 degrees C. of a temperature of the ambient air.

In one embodiment, providing the cooled air to a user includes providing the cooled air to passengers in a mass-transit vehicle.

According to another aspect of the invention, an air treatment system for supplying air to a vehicle having a passenger cabin includes: an air collection system for collecting air from one of outside the passenger cabin or inside the passenger cabin; a first air pathway for receiving air collected by the air collection system; a heating section operatively coupled to the first air pathway and configured to elevate a temperature of the ambient air in the first air pathway to a first prescribed temperature that deactivates airborne pathogens; a cooling section operatively coupled to the first air pathway and configured to reduce the temperature of the ambient air heated by the heating section to a second prescribed temperature, the second prescribed temperature lower than the first prescribed temperature; and a ventilation circuit coupled to the first air pathway and configured to provide the cooled air to the passenger cabin.

In one embodiment, the vehicle is a car, bus, truck, train, or aircraft.

According to another aspect of the invention, an air treatment system for supplying air to a building having an interior space includes: an air collection system for collecting air from one of outside the interior space or within the interior space; a first air pathway for receiving air collected by the air collection system; a heating section operatively coupled to the first air pathway and configured to elevate a temperature of the ambient air in the first air pathway to a first prescribed temperature that deactivates airborne pathogens; a cooling section operatively coupled to the first air pathway and configured to reduce the temperature of the ambient air heated by the heating section to a second prescribed temperature, the second prescribed temperature lower than the first prescribed temperature; and a ventilation circuit coupled to the first air pathway and configured to provide the cooled air to the interior space.

To the accomplishment of the foregoing and related ends, the invention, then, comprises the features hereinafter fully described and particularly pointed out in the claims. The following description and the annexed drawings set forth in detail certain illustrative embodiments of the invention. These embodiments are indicative, however, of but a few of the various ways in which the principles of the invention may be employed. Other objects, advantages and novel features of the invention will become apparent from the following detailed description of the invention when considered in conjunction with the drawings.

### BRIEF DESCRIPTION OF DRAWINGS

In the annexed drawings, like references indicate like parts or features.

FIG. 1 is a simple schematic diagram illustrating an exemplary breathing system in accordance with an embodiment of the invention.

FIG. 2 illustrates a detailed schematic view of an exemplary breathing system in accordance with an embodiment of the present invention.

FIG. 3 is a graph showing virus life (SARS corona virus (CoV-2)) with respect to temperature.

FIG. 4 is a schematic diagram illustrating exemplary heating modules that can be used in the breathing apparatus according to the invention.

FIG. 5 is a graph illustrating the temperature rise vs. time for an exemplary PTC device powered by a 24 VDC battery.

FIG. 6 illustrates a detailed schematic view of another exemplary breathing system in accordance with an embodiment of the present invention.

FIG. 7 is a graph illustrating temperatures produced by the heating section and cooling section of the device according to the invention.

FIG. 8 is a flow chart illustrating exemplary steps of a method for deactivating an airborne pathogen in accordance with the present invention.

FIG. 9 is a schematic diagram illustrating a vehicle having an exemplary air purification system in accordance.

FIG. 10 is a schematic diagram illustrating building having an exemplary air purification system in accordance with the invention.

### DETAILED DESCRIPTION OF INVENTION

Embodiments of the present invention will now be described with reference to the drawings, wherein like reference numerals are used to refer to like elements throughout. It will be understood that the figures are not necessarily to scale.

The invention will be described in the context of a breathing apparatus (e.g., a respirator) for an individual. However, other applications can benefit from the device and method described herein. For example, aspects of the invention may be applied to military breathing devices, heating, ventilation and air-conditioning (HVAC) systems for use in homes and commercial buildings (e.g., hotels, hospitals, apartments, etc.) and mobile use (e.g., in automobiles and public transportation, including busses, trucks, trains, and aircraft).

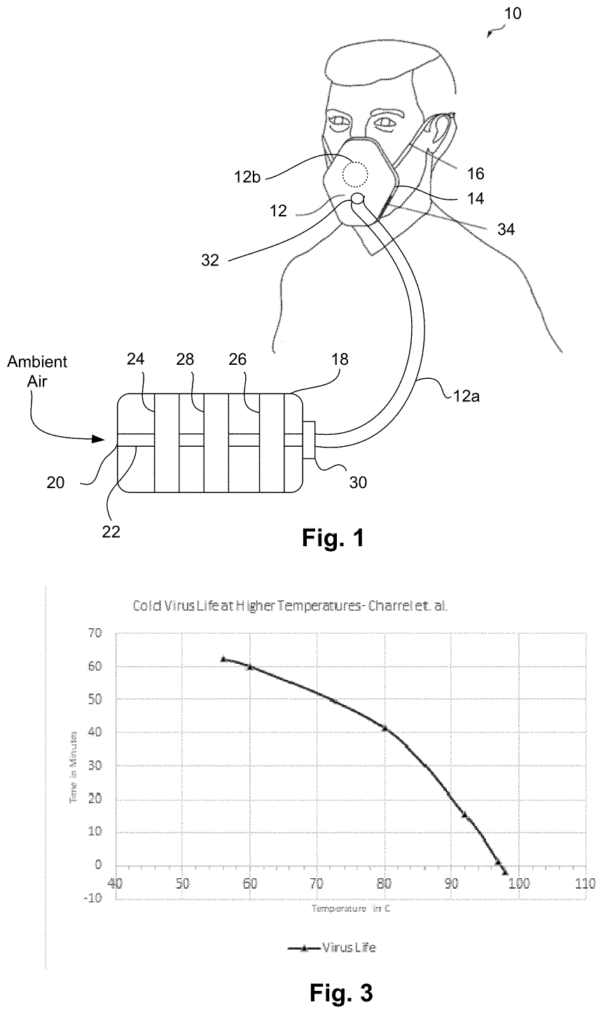

Referring to FIG. 1, illustrated is an exemplary breathing apparatus 10 according to embodiments of the invention. The breathing apparatus 10 includes a facemask 12 which is sized and shaped to fit over a person's face, covering the mouth and/or nose of the wearer. Facemask 12 may be rigid or flexible and may include a flexible sealing ring 14 around the outside which forms a partial or full seal with the wearer's face. A strap, string 16 or other suitable structure securely holds the facemask 12 on the wearer's face. The facemask includes a breathing tube 12a (also referred to as a second air pathway 12a) configured to receive air from a conditioning module as discussed in more detail below. While the second air pathway 12a is shown external to the facemask 12, portions of the second air pathway 12a be continue inside the facemask 12 to a breathing port 12b, which provides conditioned air to the user.

The breathing apparatus 10 further includes a conditioning module 18 that conditions the ambient air. As will be described in further detail below, the conditioning module 18 deactivates airborne pathogens in the air by heating the air to a high temperature, and then cools the air to a level comfortable for a user to breath. As used herein, the term “pathogen” is defined as an infectious microorganism or agent, such as a virus, bacterium, protozoan, prion, viroid, fungus or the like. Further, the term “deactivate” as herein with respect to pathogens is defined as killing or otherwise rendering the pathogen harmless. The conditioning module 18 may be a portable unit that can be worn by a user, e.g., as a backpack or attached to a belt. While a portable unit is preferred, the conditioning module 18 may be fixedly mounted to a cart that can be moved with the user.

The conditioning module 18 includes an inlet 20 for collecting ambient air, the inlet 20 coupled to and providing the collected ambient air to a first air pathway 22 that channels the ambient air through the conditioning module 18. The conditioning module 18 includes a heating section 24 operatively coupled to the first air pathway 22 to receive a flow of ambient air. The heating section 24 elevates a temperature of the ambient air to a first prescribed temperature. The first prescribed temperature is a temperature sufficiently high to deactivate airborne pathogens in the collected ambient air (e.g., between 170 degrees C. and 190 degrees C.

The conditioning module 18 also includes a cooling section 26 operatively coupled to the first air pathway 22 and downstream from the heating section 24. The cooling section 26 is configured to cool the heated air to a second prescribed temperature that is lower than the first prescribed temperature. The second prescribed temperature is a temperature that is comfortable to breathe, preferably within 3 degrees C. of the temperature of the ambient air.

Optionally, the conditioning module 18 includes one or more filter sections 28 to filter the ambient air. In the exemplary embodiment shown in FIG. 1 the filter section 28 is arranged downstream from the heating section 24 and upstream from the cooling section 26. It should be appreciated, however, that the filter section 28 may be arranged in other locations relative to the heating and cooling sections, and either one or more than one filter sections may be utilized. For example, one filter section may be arranged upstream from the heating section 24 to filter ambient air provided to the heating section, one filter section may be located downstream from the cooling section 26 to filter air after it has been cooled, and one filter section may be arranged between the heating section 24 and the cooling section 26 to filter the heated air provided to the cooling section.

Once the air has been cooled, it is communicated from the first air pathway 22 to the second air pathway 12a, where it flows to the facemask 12. A coupler 30 may be included with the facemask 12, for example, at one or both ends of the second air pathway 12a. The coupler 30 enables the facemask 12 to be selectively connected to the conditioning module 18. This can be advantageous for cleaning and/or replacement of the facemask 12, the conditioning module 18 and/or the air pathway 12a.

A first one-way valve 32, such as a check valve or the like, is coupled to the end of the second air pathway 12a and allows conditioned air from the conditioning module 18 to enter the facemask 12, but prevents air exhaled by the user from entering the second air pathway 12a. Thus, the first one-way valve 32 inhibits mixing of air exhaled by the user with air in the second air pathway 12a. A second one-way valve (not shown) is coupled to an exhaust port 34 of the facemask 12 for venting exhaled air to the ambient environment, the second one way valve preventing air from entering the facemask 12 through the exhaust port 34 as the user inhales, but enables air to be expelled from the exhaust port 34 as the user exhales.

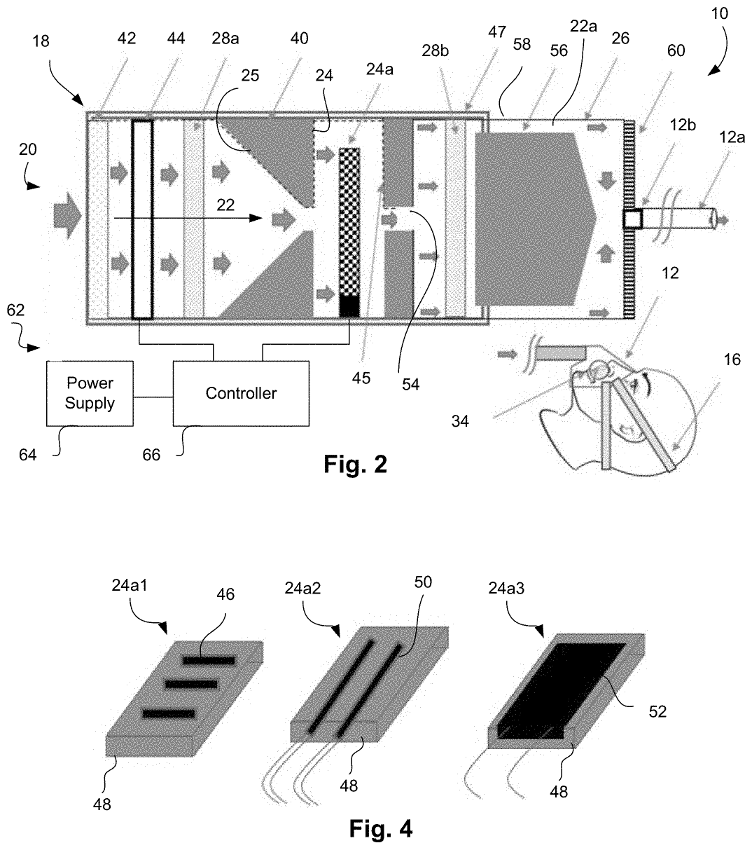

Referring to FIG. 2, an embodiment of the breathing apparatus 10 in accordance with the invention is shown in more detail. The exemplary breathing apparatus may include five sections (heating, electronics and power, cooling, breathing tube and facemask). Ambient air enters the conditioning module 18 through inlet 20 of enclosure 40 and passes through a dust filter 42. The dust filter 42 removes relatively large-size contaminants, which if permitted to enter the conditioning unit 18 can, over time, inhibit air flow through the filter sections 28a, 28b (discussed below). Ambient air is pulled into the first air pathway 22 of the enclosure 40 by fan 44 (e.g., an electric fan), which is arranged within the first air pathway 22. The first air pathway 22 may extend from the air inlet 20 to the output of the cooling section 26. The fan 44 is operative to create a continuous positive airway pressure between an output side of the fan 44 to the breathing port 12b. Preferably, the fan 44 creates a positive pressure of up to 1 cm of water height (3 cm of water height is the maximum pressure created by deep inhalation, for example, by an athlete). One or more temperature sensors may optionally be included at various locations within the air pathway 22 to monitor and/or control the heating and cooling sections as well as the fan speed.

Ambient air propelled by the fan 44 passes through a first filter section 28a in the first air pathway 22, the first filter section 28a located upstream from the heater section 24. The filter section 28a is configured to filter the ambient air provided to the heating section 24. In this regard, the filter section 28a may comprise a micro-particle filter element configured to block 0.05 micron airborne pathogens. As will be appreciated, other types of filter elements may be used with different filtering capabilities, and reference to a 0.05 micron filter is exemplary. Additionally, the filter element 28a may include a hydrophobic coating to capture liquid in the air.

The filtered ambient air then is directed into the heater section 24 located within the enclosure 40. The enclosure 40 may be formed from various materials, including plastic (e.g., high-temperature-withstanding plastic made of polysulfone, PEEK, PTFE [Teflon], etc.), metal (e.g., copper, aluminum) and the like. In one embodiment, the enclosure 44 is formed from plastic with a metal inner-liner formed from copper or aluminum. The metal inner-liner helps to reduce pathogen life on the inner surfaces of the conditioning unit 18, particularly before the heater. In another embodiment, low viral adhesion plastic material may be used instead of or in combination with the metal inner-liner. An exterior of the enclosure 40 may be covered by an outer metal enclosure (preferably copper or aluminum) to minimize electromagnetic radiation generated by the breathing apparatus 10 so as to minimize interference to nearby equipment per proven international safety standards for medical equipment (e.g., IEC 60601).

An input 25 to the heating section 24 of the enclosure 24a may have a funnel shape to focus the flowing air along a central portion of the heating element 24a. In this manner, the air can be rapidly and evenly heated to the first prescribed temperature. The funnel-shaped entry into the heating section 24 serves to minimize the space around a heating element 24a such that the filtered air achieves maximum contact with the heating element or is in very close proximity to the heating element as the air passes through the heating section 24.

Portions 45 of the air pathway 22 after the heater element 24a can be lined with a thin layer of aluminum (<0.060″ thick) and connected to an outer metal cover 47 to dissipate the heat and help cool the heated air. An optional temperature sensor located in the air pathway 22 after the heater section 24 can be used to monitor the temperature of the heated air and/or to provide closed-loop temperature regulation (which may be implemented by a controller, discussed below). The heating section 24 is preferably configured such that it can be cleaned and disinfected without the use of a tool to open the heater section. Alternatively, a metal housing without a plastic enclosure can be used for the heating section enclosure to minimize pathogen life on a plastic surface.

As noted above, the air is heated to a temperature sufficient to deactivate airborne pathogens in the collected air. Referring briefly to FIG. 3, illustrated is a graph showing the COVID-19 virus life versus temperature. The graph is obtained by fitting data provided in Charrel, et al. (Evaluation of heating and chemical protocols for inactivating SARS-CoV-2) with a non-linear quadratic regression equation (Time, y=ax2+bx+c, with a=−0.039, b=4.53 and c=−71.25, where x is Temperature) and extrapolating to estimate reduced virus life in contact-times and/or the temperature required to deactivate the virus in less than one minute, which produces an operating temperatures of equal to or greater than 98±1° C. It is possible, at even higher air temperatures, e.g., approximately 120° C., which is roughly 20-25% greater than 98° C., that the virus life can be reduced instantaneously, e.g., in a sub-second, based on the airborne pathogen contact time with the heater. This can be achieved with an air heater at 1.5-2.0 times the air-contact temperature, e.g., with a heater capable of operating between 180-240° C. or higher.

In view of the above, a heating surface of the heating element 24a over which the ambient air flows is preferably maintained at a temperature within the range of 200° C. to 250° C. Such temperature of the heating surface can raise the temperature of the incoming air to the first prescribed temperature, which preferably is between 170-190° C. As will be appreciated, the above temperatures are exemplary and other temperatures may be utilized so long as they deactivate the airborne pathogen within a reasonable time span.

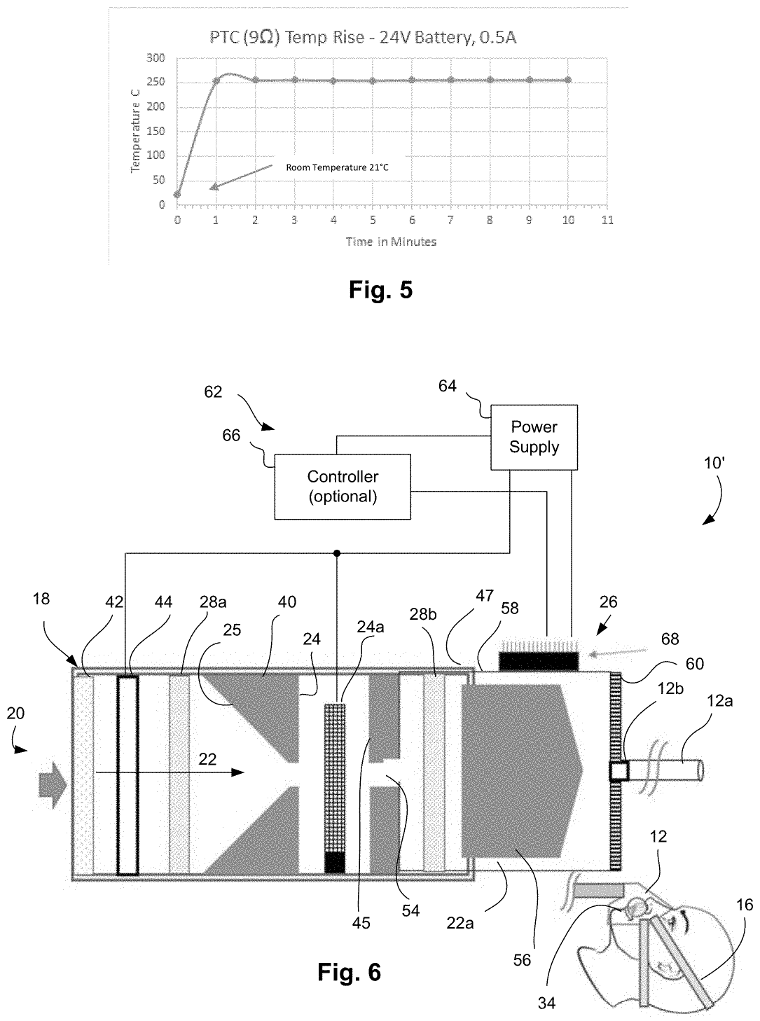

The heating element 24a, which also may be referred to as a heating block, can be formed using a heating element embedded in an aluminum casing (e.g., an anodized aluminum casing). An exemplary heating element 24a may have rough dimensions of 1″×1″×0.25″, and may include fixed or variable heating elements to maintain optimum power. Referring briefly to FIG. 4, illustrated are several exemplary heating elements that may be used in the heater section 24. In one embodiment, the heating element 24a1 is formed from one or more fixed-value resistor elements 46 embedded within the aluminum block 48, the resistor elements 46 generating heat as current passes through the resistors. In another embodiment, the heating element 24a2 is formed from one or more circular or other shaped heater cartridges 50 embedded within the aluminum block 48. In yet another embodiment, the heating element 24a3 is formed from one or more self-regulating positive temperature coefficient (PTC) elements 52 embedded with the aluminum block 48. The PTC heater elements 52 may exhibit a temperature rise time characteristic shown in FIG. 5 (using a 24 VDC battery with a 9 Ohm PTC device).

A maxim power level supplied to the heating elements 24a may be regulated to improve overall safety. More particularly, a measured temperature of the ambient air may be used to set a maximum power level supplied to the heating elements. For example, the measured ambient temperature may be compared to a base-line ambient temperature, where the base-line ambient temperature is an initial predetermined temperature, e.g., 20-22 degrees C. If the measured ambient temperature is greater than the base-line ambient temperature, then the maximum power supplied to the heating element 24a may be limited (e.g., the maximum current provided to heating element can be limited to lower the maximum possible heat output by the heating element) as the warmer ambient requires less work from the heating section. Conversely, if the actual ambient temperature is less than the base-line ambient temperature then the maximum power supplied to the heating element 24a can be increased (e.g., the maximum current provided to the heating element can be increased to increase the maximum heat output by the heating element) as the colder ambient requires more work from the heating element. An exemplary equation for determining the maximum power provided to the heating element is provided by Hp=Tb/Ta*Pr, where Hp is the calculated maximum heating element power, Tb is the base-line ambient temperature, Ta is the actual ambient temperature, and Pr is the regulated power of the heating element. By regulating the maximum power provided to the heating element 24a, the likelihood of overheating due to ambient environments having elevated temperatures is minimized, as is the likelihood of insufficient heating in ambient environments with lower temperatures.

Referring back to FIG. 2, the heated and filtered air exits the heating section 24 through a passage 54. In one embodiment, the passage 54 is dimensioned to restrict an amount of airflow through the heater section 24, thereby maximizing the time the air is in contact with the heating element 24a. Upon exiting the passage 54, the heated air enters the cooling section 26, which cools the heated air to near ambient temperature. The process by which the air is cooled is conduction through the thin walls of the cooling section 26, which communicates the internal heated air to the external ambient air. The cooling section 26 includes a second filter element 28b (e.g., medical grade micro-particulate filter) arranged in the first air pathway 22 downstream from the heating section 24 and upstream from the facemask portion 12. The second filter element 28b helps to trap 0.05 micron diameter and larger airborne pathogens that may not have been deactivated by the heater section 24. The second filter element 26, in addition to a hydrophobic coating, can also include a coating that includes cationic moieties, which will help to trap any anionic viral ribonucleiacid (RNA) fragments resulting from the destruction of the viral core particles in the air path, due to exposure to the high temperature heater section 24. As is known, cationic moieties are positively charged, ionic groups, e.g., NH₄ ⁺.

The cooling section 26 includes an air diverter 56 arranged within a thin metal-walled housing 58 (<0.06″ thick, preferably aluminum or copper). The air diverter's function is to direct the air exiting the heating section 24 to travel adjacent to the thin heat-expelling wall (e.g., aluminum wall) of the cooling section 26 (e.g., to create a thin flow path that maximizes contact of the air with the wall). In one embodiment, the diverter 56 defines narrow channels 22a along an inner surface of the housing walls, which maximizes the contact of air flowing through the cooling section 26 with the walls of the housing 58, thereby providing maximum cooling effect. A heat extractor (het sink) 60 can be arranged on one or more outer surfaces of the housing 58 to further improve extraction of heat from the air.

The air diverter 56 can be formed from a temperature-resistive plastic material a metal material, or other material that minimizes or inhibits pathogen life and does not tend to raise the absorb heat. Any heat that is absorbed by the diverter 56 can be transferred via direct exposure e.g., by a thermoplastic material or by direct connection to a heat expeller to the ambient surroundings and/or indirectly via the cooling section wall or even the metal casing of the heating section 24. For example, the thin metal housing 56 of the cooling section 26 may be connected to the outer metal casing of the heating section 24 to further expel heat (and improve system electromagnetic interference (EMI)).

The cooled air exits the cooling section 26 and travels to the facemask 12 via the second air pathway 12a. A coupler 12b enables the second air pathway 12a and facemask 12 to be decoupled from the conditioning module 18 for maintenance purposes and/or repair purposes. While one coupler is shown, multiple couplers may be utilized, e.g., an additional coupler may be arranged on the facemask 12 to enable the second air pathway 12a to be decoupled from the facemask 12. The facemask 12 and second air pathway 12a may be a medical grade, one-time use device or a reusable device. Valves within the facemask 12 prevent mixing of exhaled air with the incoming freshly filtered, pathogen-deactivated clean air. The facemask can be configured to cover the full head, partially cover the head and cover the full face or partially cover the face.

The breathing system 10 further includes an electronics section 62 for powering and/or controlling the system 10. The electronics section 62 includes a power supply 64 electrically coupled to the fan 44, the heating section 24 and/or the cooling section 26 (in the embodiment in which the cooling system includes an active cooler, as discussed below), the power supply 64 operative to provide electric power to the fan, heating section and/or cooling section. In the preferred embodiment, the power supply 64 includes a battery having sufficient size to provide power over a prescribed time interval, e.g., 8 hours. However, it is contemplated that the power supply 64 may be connected to a wall output or the like. Connection to a wall outlet has the benefit of allowing continued operation in the event the battery is depleted and/or to charge the battery.

The battery can be a 24V DC battery having sufficient ampere-hour (AH) capacity (e.g., 6 AH, model no. VIDAR-1826240002 weighing 1.21 lbs. or 10.4 AH, model no. VIDAR-1826240006 weighing 2.42 lbs.) to preferably support 8 hours continuous operation. As will be appreciated, batteries having different voltages and or AH ratings may be employed.

Optionally, the electronics section 62 may also include a controller 66 operatively coupled to the fan 44, the heating section 24, and/or the cooling section 26. The controller 66 may include a processor and memory for executing computer instructions. In this regard, the controller 62 may implement temperature control of the heating section 24 and the cooling section 26 and flow control (fan speed) to ensure the prescribed air temperatures are obtained at the output of each respective section. Power from the power supply 64 may be provided to the various sections of the conditioning module 18 through the controller 66, or via direct connection to the power supply. The electronics section 62 and the conditioning module 18 can be contained within a pouch that is wearable by the user (e.g., along the side or on the back). Straps and buckles can be used to secure the pouch to the user.

By directing post-filtered air through the heater section 24, high air temperatures can be achieved sufficient to deactivate the airborne pathogens. Further, by cooling the post-heated air with a heat expeller (cooling section 26), the air temperature traversing the breathing circuit can be reduced to arrive close to ambient temperatures, prior to subject inhalation. As used herein, the breathing circuit includes the second air pathway 12a and the mask 12.

Moving now to FIG. 5, illustrated is breathing apparatus 10′ in accordance with another embodiment of the invention. The breathing apparatus 10′ of FIG. 5 is highly similar to the breathing apparatus 10 of FIG. 2, and therefore only differences between the two embodiments are discussed below.

The electronics section 62 includes a 24V, 6200 mAH (6.2 ΔH) lithium ion battery power source to support 8 hour continuous operation, and a light or a LED to demonstrate ON mode, battery status and a ON-OFF switch. The heating section 24 utilizes PTC heating elements to heat the incoming air. Since the PTC heating elements are self-regulating, a sensor-based feedback control may not be necessary (thus possibly eliminating the controller 66 and simplifying the design).

Cooling section 26 includes a thermoelectric cooling module 68 (Peltier effect device) connected to the thin aluminum walled housing (<0.06″ thick) and the diverter in addition to the couplings to the heating and breathing circuit sections. The thermoelectric cooling module 68 is powered by the power supply 64. In one embodiment, controller 66 receives a temperature feedback from a temperature sensor (not shown) located in the cooling section 26 and controls the thermoelectric cooling device 68 to achieve a target temperature. In another embodiment, the thermoelectric cooling device is self-regulating without external control by the controller 66. Optionally, the diverter 56 and the thin metal wall 58 of the cooling section 26 can also be actively cooled with the thermoelectric cooling module 68 (at the expense of additional power draw from the battery). The thermoelectric cooling module 68 in combination with a heat sink 60 may or may not be used. If used, it may be connected to the diverter, outer aluminum housing of the cooling section and or the heating element. Accordingly, in contrast to the passive cooling section of the embodiment of FIG. 2, the cooling section of the embodiment in FIG. 5 is active.

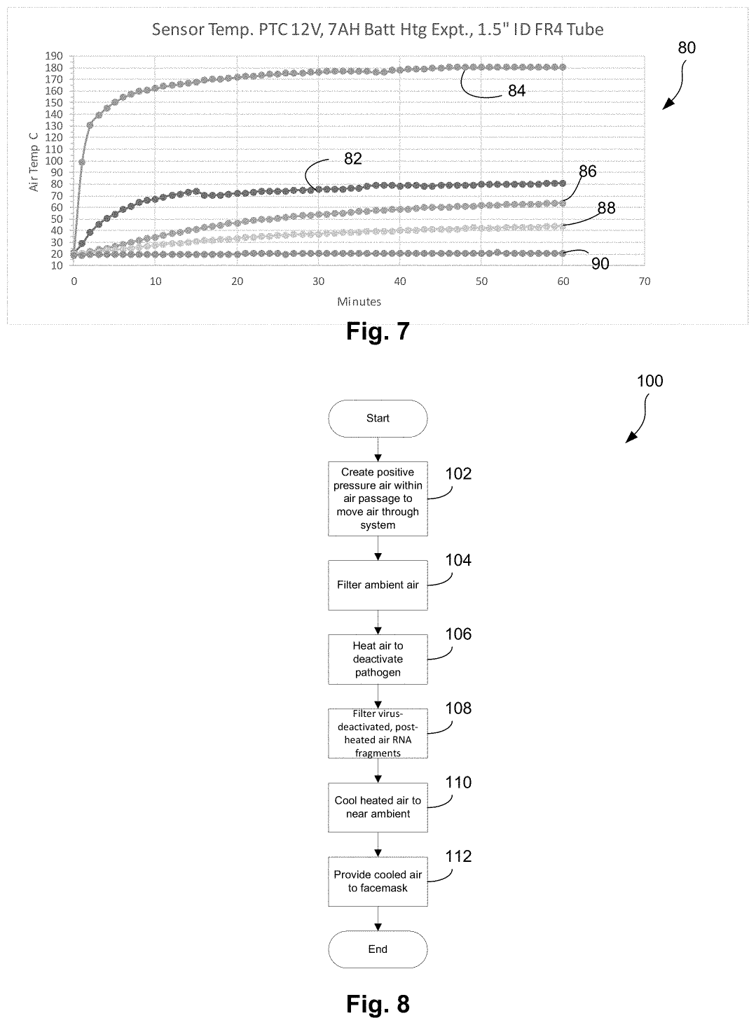

Moving to FIG. 7, illustrated is a graph 80 showing performance of various sections of a prototype breathing apparatus according to the invention. The data was obtained using a 12 volt, 7 AH battery, with an 8.4 Ohm heating resistor. A 60 mA 12 volt fan was utilized to achieve over 2.2 m/s airflow. Ambient air temperature is 20 degrees C. Temperature sensors were arranged at the input side of the heating section 24, the output side of the heating section 24, the input side of the cooling section 26, the output side of the cooling section 26, and within the breathing tube 12a. As will be appreciated, the performance of the system can be improved by using different components. For example, heating elements with higher ratings, PTC heating elements, and/or a battery having a higher voltage rating can decrease the time required to heat the air and/or can raise the maximum attainable air temperature. Further, a thermoelectric cooling device can be used in the cooling section 26 to more quickly bring the temperature down to the temperature of ambient air, if needed.

As seen in FIG. 7, curve 82 represents the temperature of air on the input side of the heating section 24, where the air temperature is already significantly raised over ambient, reaching a steady state temperature of about 70 degrees C. Curve 84 represents the temperature at the output side of the heating section 24, where the air temperature surpasses 120 degrees C. in a couple of minutes and reaches a steady state of 180 degrees C. within only a few minutes of operation.

The heated air enters the input side of the cooling section 26 and drops in temperature, as seen by curve 86. As the air passes through the cooling section 26, it continues to drop, as shown by curve 88, and enters the breathing tube (second air pathway 12a). At approximately two feet from the exit of the cooling section 26, the air temperature in the breathing tube 12a has dropped to approximately the temperature of the ambient air, as seen by curve 90.

Accordingly, the breathing apparatus in accordance with the invention can rapidly raise air temperature to deactivate airborne pathogens, and then rapidly cool the air to a temperature that is comfortable to breath.

Referring now to FIG. 8, illustrated is a flow diagram that depicts an exemplary method 100 of deactivating an airborne pathogen in accordance with the invention. Although the method descriptions and flow diagram may show specific orders of executing steps, the order of executing the steps may be changed relative to the order described. Also, two or more steps described in succession may be executed concurrently or with partial concurrence. One or more of the described or illustrated steps may be omitted.

Beginning at step 102, positive pressure is generated within an air pathway 22 of a conditioning unit 18. Such positive pressure may be generated, for example, via a fan 44 located within the air pathway 22. In generating the positive pressure, the fan 44 draws ambient air into the air pathway 22 so as to collect the ambient air, where it is filtered as indicated at step 104. Such filtering preferably utilizes a medical-grade HEPA filter capable of filtering objects larger than 0.05 microns, and more preferably capable of filtering objects larger than 0.01 microns. Filtering may further include using a hydrophobic coated filter element to trap any airborne liquid in the ambient air.

Next at step 106, the collected and filtered air is heated to a first prescribed temperature, the first prescribed temperature sufficient to deactivate airborne pathogens. Preferably, the first prescribed temperature is 190 degrees C. or higher. A heating element, such as a resistive heating element or a PTC heating element may be used to raise the temperature of the air to the first prescribed temperature. The heated air then is filtered again as indicated at step 108. In filtering the heated air, the filter element, in addition to having a hydrophobic coating, may also be coated with cationic moieties. The cationic moieties help trap any anionic RNA fragments produced as a result of deactivation of the pathogen from the heating process.

Moving next to step 110, the heated air is cooled by the cooling section 26 to bring the air temperature back down to a second prescribed temperature that is lower than the first prescribed temperature, e.g., a temperature that is comfortable to breath. Preferably, the second prescribed temperature is within 3 degrees C. of the ambient air temperature. Such cooling may be performed using thin metal housing that conducts heat from the air to the ambient surrounds. One or more heat sinks may be attached to the housing, and/or a thermoelectric device may be attached to the housing to further improve the cooling performance. The cooled air then exits the cooling section and moves to the facemask 12 through breathing tube 12a (a second air pathway), where further cooling takes place in the air tube.

Accordingly, a user is provided with conditioned air that is free of active airborne pathogens. The portable nature of the device in accordance with the invention enables the user to move freely about any space, without the need to be tethered to an air supply system.

With reference now to FIG. 9, illustrated is another aspect of the invention directed to a ventilation system for a vehicle. In FIG. 9, the conditioning module 18 is utilized with a vehicle 100, such as an automobile, although other types of vehicles, such as trucks, busses, trains, aircraft, and the like, are contemplated. As is conventional, an air collector 102 collects air from the ambient environment. Such air collector may be arranged under the dash of the automobile and selectively coupled to the cowl of the automobile (fresh air mode) or to the internal cabin of the automobile (recirculation mode) as is conventional. The air collector 102 outputs the collected air to a conditioning module 18 as described herein. The conditioning module 18 proceeds to filter, heat and cool the collected air as described herein to deactivate airborne pathogens. The conditioning module 18 then provides the cooled air to the cabin 104 of the automobile via vent system 106. Depending on the operational mode of the system, the cabin air may be exhausted through air exhaust system 108, or recirculated by the air collection system as is conventional.

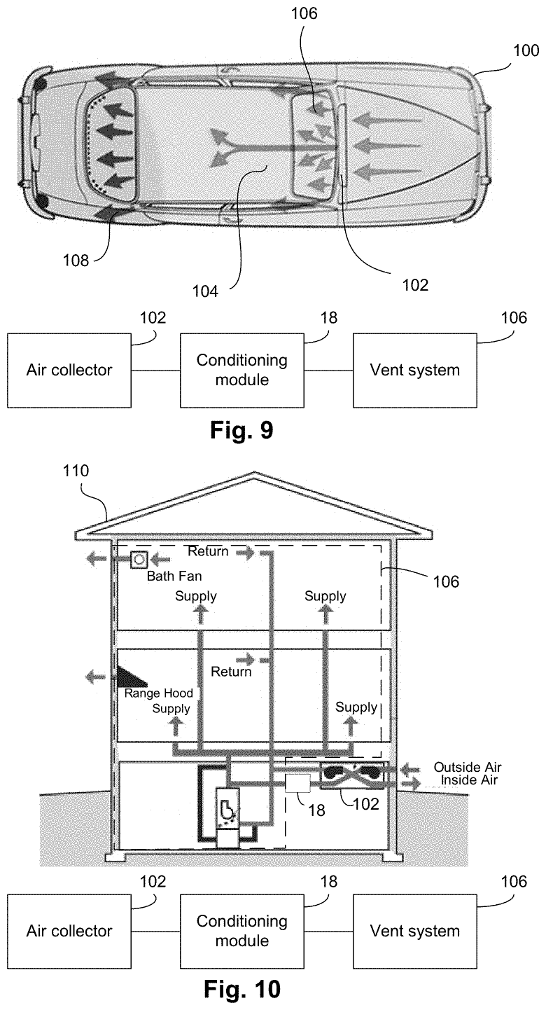

Moving to FIG. 10, illustrated is another aspect of the invention directed to a ventilation system for a building 110. In FIG. 10, the conditioning module 18 is utilized with a building 110, such as a house, although other types of buildings, such as apartments, stores, hospitals, hotels and the like, are contemplated. As is conventional, an air collector 102 collects air from the ambient environment. The air collector 102 outputs the collected air to a conditioning module 18 as described herein. The conditioning module 18 proceeds to filter, heat and cool the collected air as described herein to deactivate airborne pathogens. The conditioning module 18 then provides the cooled air to the interior space of the building via vent system 106.

Although the invention has been shown and described with respect to a certain embodiment or embodiments, equivalent alterations and modifications may occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein exemplary embodiment or embodiments of the invention. In addition, while a particular feature of the invention may have been described above with respect to only one or more of several embodiments, such feature may be combined with one or more other features of the other embodiments, as may be desired and advantageous for any given or particular application.

1. A breathing apparatus for deactivating airborne pathogens, comprising:
a first air pathway for receiving ambient air and channeling the air through a portion of the breathing apparatus;
a heating section operatively coupled to the first air pathway and configured to elevate a temperature of the ambient air in the first air pathway to a first prescribed temperature that deactivates airborne pathogens;
a cooling section operatively coupled to the first air pathway and configured to reduce the temperature of the ambient air heated by the heating section to a second prescribed temperature, the second prescribed temperature lower than the first prescribed temperature; and
a breathing circuit coupled to the first air pathway and configured to provide the cooled air to a user.

2. The breathing apparatus according to claim 1, wherein the breathing circuit comprises a facemask portion configured to cover a portion of a user's face, the facemask portion including a second air pathway configured to receive the air from the first air pathway that is cooled by the cooling section and provide the cooled air to at least one breathing port arranged to overlie a user's facial cavity.

3. The breathing apparatus according to claim 2, wherein the facemask portion further comprises:
an exhaust port configured to vent exhaled air to the ambient environment; and
at least one valve coupled to the second air pathway, the at least one valve configured to inhibit mixing of air exhaled by the user with air in the second air pathway.

4. The breathing apparatus according to claim 2, wherein the facemask portion comprises a coupler operative to selectively couple the second air pathway to the first air pathway.

5. The breathing apparatus according to claim 1, further comprising a filter element arranged in the first air pathway upstream from the heating section, the filter element configured to filter the ambient air provided to the heating section.

6. The breathing apparatus according to claim 1, further comprising a filter element arranged in the first air pathway downstream from the heating section and upstream from the breathing circuit, the filter element configured to filter the air heated by the heating section.

7. The breathing apparatus according to claim 6, wherein the filter element comprises a coating including cationic moieties.

8. The breathing apparatus according to claim 5, wherein the filter element comprises a hydrophobic coating.

9. The breathing apparatus according to claim 5, wherein the filter element comprises a micro-particle filter element configured to block 0.05 micron airborne pathogens.

10. The breathing apparatus according to claim 1, wherein the heating section includes a heating surface over which the ambient air flows, the heating section configured to maintain a temperature of the heating surface within the range of 200 degrees C. to 250 degrees C.

11. The breathing apparatus according to claim 1, wherein the first prescribed temperature is at least 190 degrees C.

12. The breathing apparatus according to claim 1, wherein the second prescribed temperature is within 3 degrees C. of a temperature of the ambient air.

13. The breathing apparatus according to claim 1, further comprising a power supply electrically coupled to at least one of the heating section and the cooling section, the power supply operative to provide electric power to the at least one of the heating section and the cooling section.

14. The breathing apparatus according to claim 13, wherein the power supply comprises a battery electrically coupled to at least one of the heating section and the cooling section.

15. The breathing apparatus according to claim 1, wherein at least one of the first air pathway or the second air pathway comprises a metal inner liner.

16. The breathing apparatus according to claim 15, wherein the metal inner liner is formed from copper or aluminum.

17. The breathing apparatus according to claim 1, wherein the heating section comprises a fan operative to create a positive pressure from an input side of the heating section to an output side of the breathing circuit, and a temperature sensor operative to measure a temperature of air output by the heating section.

18. The breathing apparatus according to claim 1,
further comprising a controller operatively coupled to at least one of the heating section and the cooling section, the controller configured to regulate at least one of a temperature of air output by the heating section and a temperature of air output by the cooling section.

19. The breathing apparatus according to claim 1, wherein the cooling section comprises a heat sink.

20. The breathing apparatus according to claim 1, wherein the cooling section comprises a thermoelectric cooling module.

21. The breathing apparatus according to claim 1, wherein the heating section comprises at least one of a resistor, a cartridge heating element, or a positive temperature heating coefficient heating element.

22. The breathing apparatus according to claim 1, wherein the facemask portion comprises a securement strap for securement of the facemask portion to the user's face.

23. A method of conditioning air provided to a user, comprising:
collecting air from the ambient environment;
heating the collected ambient air to a first prescribed temperature, the first prescribed temperature sufficient to deactivate airborne pathogens;
cooling the heated air to a second prescribed temperature, wherein the second prescribed temperature is lower than the first prescribed temperature; and
providing the cooled air to the user.

24. The method according to claim 23, wherein providing the cooled air to the user includes providing the cooled air to a facemask worn by the user.

25. The method according to claim 23, further comprising using a hydrophobic filter element to filter air provided to the user.

26. The method according to claim 23, further comprising using a filter element coated with cationic moieties to filter air provided to the user.

27. The method according to claim 23, wherein the first prescribed temperature is at least 190 degrees C.

28. The method according to claim 23, wherein the second prescribed temperature is within 3 degrees C. of a temperature of the ambient air.

29. The method according to claim 23, wherein providing the cooled air to a user includes providing the cooled air to passengers in a mass-transit vehicle.
